(19) United States
(12) Patent Application Publication
(10) Pub. No.: US 2014/0188037 A1
(43) Pub. Date: Jul. 3, 2014

(54) Method and Apparatus for Cold Plasma Bromhidrosis Treatment

(71) Applicant: Cold Plasma Medical Technologies, Inc., Scottsdale, AZ (US)

(72) Inventors: Marc C. Jacofsky, Phoenix, AZ (US); Gregory A. Watson, Sanford, FL (US)

(73) Assignee: Cold Plasma Medical Technologies, Inc., Scottsdale, AZ (US)

(21) Appl. No.: 14/145,297

(22) Filed: Dec. 31, 2013

Related U.S. Application Data
(60) Provisional application No. 61/747,868, filed on Dec. 31, 2012.

(51) Int. Cl. A61M 35/00; A61F 7/00

(52) U.S. Cl. CPC A61M 35/003; A61F 7/0085
USPC 604/23

(57) ABSTRACT

A cold plasma device for bromhidrosis treatment is described. A dielectric barrier discharge device is formed by an electrode disposed adjacent to a dielectric barrier, with the dielectric barrier being configured to apply a cold plasma to a bromhidrosis treatment surface. The electrode is coupled to a pulsed high voltage cold plasma power supply, which may be external or internal to the body containing the DBD device. The cold plasma bromhidrosis treatment device can be powered by batteries or by an AC/DC adaptor.

### CROSS REFERENCE TO RELATED APPLICATIONS

This application claims the benefit of U.S. Provisional Application No. 61/747,868, filed Dec. 31, 2012 and entitled “Method and Apparatus for Cold Plasma Bromhidrosis Treatment,” which is incorporated herein by reference in its entirety.

This application is related to U.S. Provisional Application No. 60/913,369, filed Apr. 23, 2007; U.S. patent application Ser. No. 12/038,159, filed Feb. 27, 2008 (which issued as U.S. Pat. No. 7,633,231); U.S. patent application Ser. No. 13/620,118, filed Sep. 14, 2012, and Attorney Docket No. 3022.0070001, entitled “Method and Apparatus for Dielectric Barrier Discharge Wand Cold Plasma Device,” to be filed on Dec. 31, 2013, each of which are herein incorporated by reference in their entireties.

### BACKGROUND

1. Field of the Art

The present disclosure relates to devices and methods for cold plasma generation, and, more particularly, to such devices and methods for cold plasma bromhidrosis treatment.

2. Background Art

According to Euromonitor International, a market research firm, $2.3 billion was spent on deodorant and antiperspirant in 2006, just in the United States. Traditionally, people apply a deodorant and/or antiperspirant at least once per day to prevent, reduce, or cover up the amount of offensive odor created in the moist environment of the human underarm. Deodorants do not necessarily reduce sweat production but do reduce odor formation while antiperspirants actually reduce sweat production. Both deodorants and antiperspirants are chemical or pharmacological in their makeup and safety concerns have been raised over the repeated application of these compounds to sensitive tissues over long periods of time. Both deodorants and antiperspirants serve to reduce odor formation but through different mechanisms. Deodorants create an inhospitable environment for microbes while antiperspirants reduce the moisture that contains a major food source for microbes. So while the mechanism of action differs, the end goal is to reduce microbe production and metabolism in the underarm region. Cold plasmas are known to reduce microbial activity and also denature organic molecules. Therefore, cold plasma application to the underarm can reduce the development of microbes as well as reduce existing organic odors.

The majority of human sweat is made from water, with smaller amounts of urea, salts, sugars, and ammonia. Bacteria and yeast populations flourish in the warm and moist conditions found in the moist underarm region and, in varying combinations, are the sources of typical underarm odor.

There are two types of sudoriferous or sweat glands, eccrine and apocrine. Eccrine sweat typically starts out as odorless, but this sweat does work to soften the epidermal keratin, which can itself cause odor. Bacteria can thrive in the underarm area not only for the aforementioned reasons, but also because they can feed upon the eccrine sweat and the softened keratin, thereby contributing to the malodorous condition.

Apocrine sudoriferous glands are far more limited in their anatomical distribution, located around the pectoralis muscles (breasts), in the axillae, and the groin, with a small number of these apocrine elements surrounding the eyes and ears. The apocrine sudoriferous glands generate pheromones and are primarily responsible for causing body odor, a condition known medically as bromhidrosis. During this process, apocrine sweat is broken down by Corynebacterium, a genus of Gram-positive, rod-shaped bacteria. Strong smelling short-chain fatty acids are produced when these bacteria digest and further break down the secretions from the apocrine sudoriferous glands. The most common of these fatty acids is (E)-3-methyl-2-hexanoic acid (E-3M2H), which is brought to the skin surface bound by 2 apocrine secretion odor-binding proteins, ASOB1 and ASOB2. ASOB2 has been identified as apolipoprotein D (apoD), a known member of the lipocalin family of carrier proteins.

Traditional means of deodorant protection can result in allergic or toxic reactions in some patients. Thus, it is desirable to identify other means of deodorant protection that can avoid the allergic or toxic reactions. It is further desirable that such means be painless, non-invasive and/or self-sterilizing.

### BRIEF SUMMARY OF THE INVENTION

An embodiment is described of a cold plasma bromhidrosis treatment device that includes a dielectric barrier discharge device formed by an electrode disposed adjacent to a dielectric barrier. The dielectric barrier is configured to apply a cold plasma to a bromhidrosis treatment surface. The device also has a body affixed to the dielectric barrier discharge device, where the body includes a housing to accommodate a pulsed high voltage cold plasma power supply. The electrode is coupled to the pulsed high voltage cold plasma power supply.

A further embodiment is described of a method of cold plasma bromhidrosis treatment. The method includes receiving, by a dielectric barrier discharge (DBD) device, electrical energy to generate a cold plasma, where the dielectric barrier discharge (DBD) device formed by an electrode disposed adjacent to a dielectric barrier. A body is affixed to the dielectric barrier discharge device, where the body includes a housing to accommodate a pulsed high voltage cold plasma power supply for supply of the electrical energy to the electrode. The method finally includes applying the cold plasma by the dielectric barrier to the bromhidrosis treatment surface.

### BRIEF DESCRIPTION OF THE DRAWINGS/FIGURES

FIG. 1 illustrates a schematic for a dielectric barrier discharge device for production of cold plasma.

FIG. 2 illustrates a schematic drawing of a power circuit for a cold plasma DBD deodorant device, in accordance with an embodiment of the present disclosure.

FIG. 3 illustrates a schematic drawing of a configurable electronic circuit for a power circuit for a cold plasma DBD deodorant device, in accordance with an embodiment of the present disclosure.

FIGS. 4A and B illustrate two schematic views of a cold plasma deodorant device, in accordance with an embodiment of the present disclosure.

FIG. 5 illustrates a schematic of an electrode assembly of a cold plasma DBD deodorant device, in accordance with an embodiment of the present disclosure.

FIG. 6 is a photographic illustration of a cold plasma DBD deodorant device showing the optional AC to DC converter, in accordance with an embodiment of the present disclosure.

FIG. 7 illustrates a cold plasma DBD deodorant device in use, in accordance with an embodiment of the present disclosure.

FIG. 8 provides a further illustration of a cold plasma DBD deodorant device in use, in accordance with an embodiment of the present disclosure.

FIG. 9 illustrates flowchart of a method for providing bromhidrosis treatment using a cold plasma device, according to an embodiment of the present disclosure.

### DETAILED DESCRIPTION OF THE INVENTION

Cold temperature plasmas have attracted a great deal of enthusiasm and interest by virtue of their provision of having relatively low gas plasma temperatures. The provision of plasmas at such a temperature is of interest to a variety of applications having temperature sensitive substrates, including wound healing, anti-bacterial processes, various other medical therapies and sterilization.

Dielectric barrier discharge (DBD) devices offer a high bactericidal effectiveness. It has been noted that DBD plasma offers a gentle but rapid tissue antisepsis that results in the inactivation of diverse pathogens. In addition to the bactericidal effects of DBD plasmas, they excel at denaturing proteins and other organic molecules. Direct application of DBD plasma is effective not only in destroying the bacterial loads found in the human underarm, but also in inhibiting the very mechanism by which the odors are produced. Consequently, a DBD plasma provides a very effective antiseptic without the added complications of using biocides such as chlorhexidine, iodine, or various types of alcohols. Thus, a DBD plasma provides a deodorant that is self-sterilizing, painless, non-invasive, while not resulting in an allergic or toxic reaction.

Embodiments of the present disclosure may treat one or more forms of bromhidrosis. In an exemplary embodiment, the DBD device can be a hand-held DBD device. Embodiments of the DBD device may be applied to the underarm area for reduction of bacterial load (antisepsis) with a few seconds of treatment time. Published results in the literature indicate that a DBD device can result in a 6-log reduction of bacterial load with a 5 second exposure.

In an exemplary embodiment of the preset invention, a cold plasma DBD deodorant device may contain an internal pulsed high voltage power supply, together with a dielectric barrier discharge surface that can be brought into direct contact with the underarm. The dielectric barrier surface could be made of PTFE, polyoxymethylene, crystalline quartz, and the like, together with an underlying conductive electrode having sufficient capacitance to support the dielectric discharge.

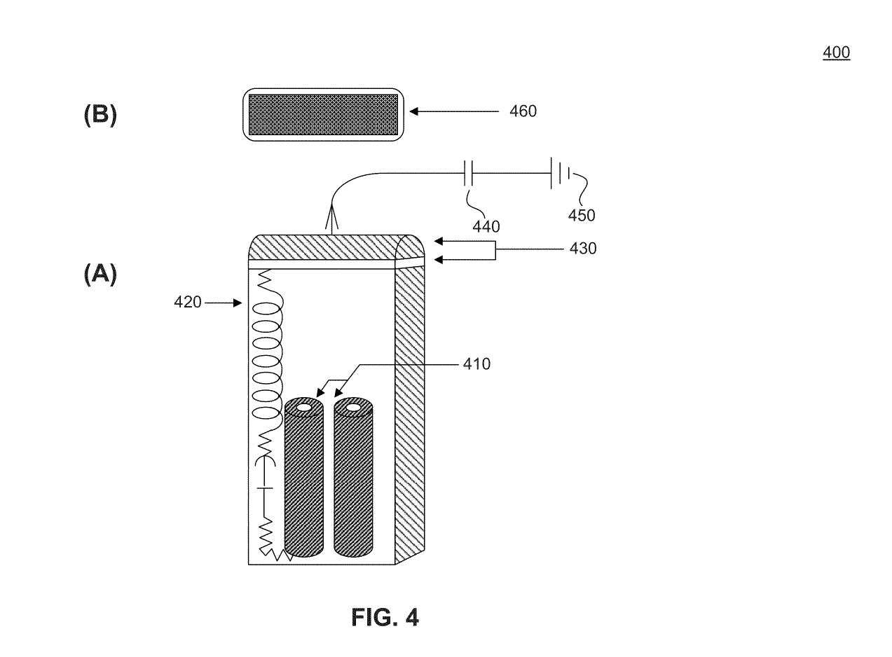

Different embodiments of the present disclosure can use different sources of electrical energy. In one embodiment, a cold plasma DBD device can be AC-powered. In an alternative embodiment, a cold plasma DBD device can be DC-powered. The size of the enclosure of a cold plasma DBD device and its duration of use would differ with the different electrical energy sources. Despite their difference in sizes, nevertheless both the AC-powered and the DC-powered embodiments may have similar form factors. FIG. 4A illustrates a battery-powered embodiment of the present disclosure. Such an embodiment may use alkaline batteries that require periodic replacement or a rechargeable battery pack, similar to an electric toothbrush.

An exemplary electrical energy input signal to the cold plasma DBD device would be a pulsed high voltage electrical signal of sufficient amplitude to provide electrical energy to the cold plasma DBD device. The pulsed high voltage electrical signal may be a single frequency electrical signal, or a multi-frequency pulsed high voltage electrical signal. Required signal amplitude may vary based on the type of pulsed high voltage electrical signal used and the properties of the selected dielectric barrier material. Further details of embodiments of the present disclosure can be found by reference to the following figures.

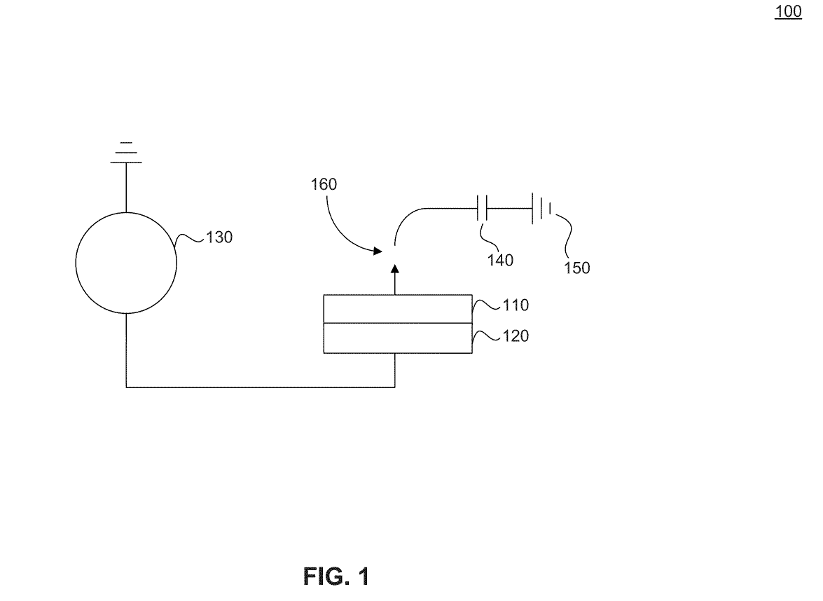

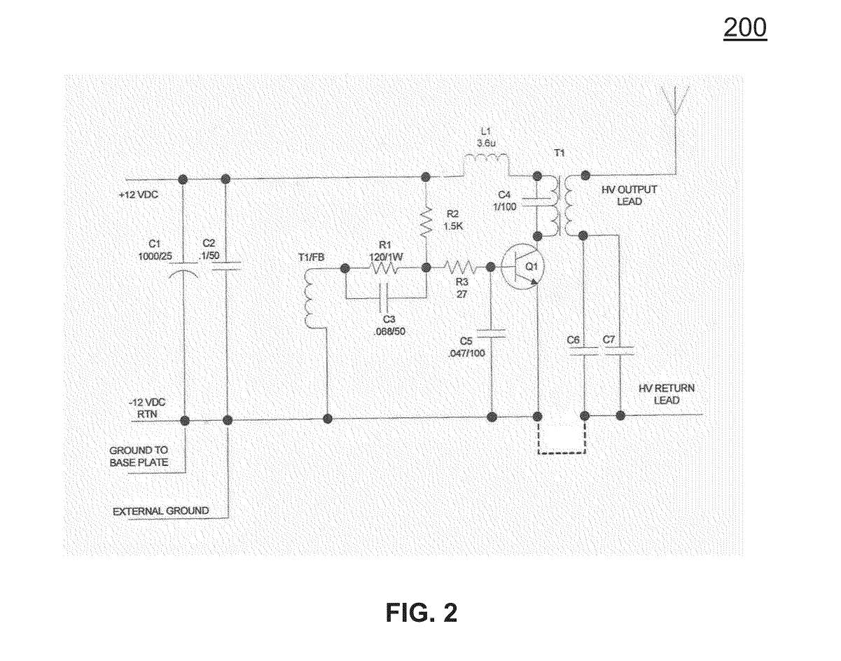

FIG. 1 illustrates a schematic for a dielectric barrier discharge device for production of cold plasma 160. As FIG. 1 illustrates, a dielectric barrier discharge (DBD) device containing one conductive electrode 120 covered by a dielectric barrier 110. The electrical return path is formed by the ground 150 provided by the target substrate undergoing the cold plasma treatment and the target substrate represented by capacitance 140. Energy for the dielectric barrier discharge device can be provided by a pulsed high voltage power supply 130, such as that described below and illustrated in FIG. 2. More generally, energy is input to the dielectric barrier discharge device in the form of pulsed electrical voltage to form the plasma discharge. By virtue of the dielectric barrier, the discharge is separated from the conductive electrode and electrode etching and gas heating is reduced. The pulsed electrical voltage can be varied in amplitude and frequency to achieve varying regimes of operation. In this embodiment, the target (e.g., user's underarm in the case of a cold plasma DBD bromhidrosis device) form a ground sink with capacitance. However, embodiments of the present disclosure are not limited to situations where the target is required to provide a ground. For example, in another embodiment, the cold plasma DBD bromhidrosis device may include an electrode with a built-in ground.

FIG. 2 illustrates a schematic drawing of a power circuit for a cold plasma DBD deodorant device An oscillator circuit is coupled to the 12 V DC power supply and the primary winding of a resonance transformer T1. The resonance transformer provides a magnification of the voltage to the secondary windings, which are in turn connected to the high voltage output connector of the power circuit. In an exemplary embodiment, the output voltage may be 7.5 kV, with a frequency of the output waveform being 40 kHz. The resulting high voltage at the output of the power circuit is determined by the magnification (turns ratio) of the transformer and can be raised to as high a level as may be reasonably desired. However, increased voltages typically result in an increased weight and size of the transformer required to output the increased voltage. FIG. 2 is merely an exemplary circuit (and therefore not limiting) for providing a pulsed high voltage cold plasma power supply for use with a cold plasma DBD deodorant device. Other approaches include, but are not limited to, those multi-frequency harmonic-rich approaches described in U.S. Provisional Application No. 60/913,369, filed Apr. 23, 2007; U.S. patent application Ser. No. 12/038,159, filed Feb. 27, 2008 (which issued as U.S. Pat. No. 7,633,231); and U.S. patent application Ser. No. 13/620,118, filed Sep. 14, 2012, all of which are incorporated by reference in their entireties.

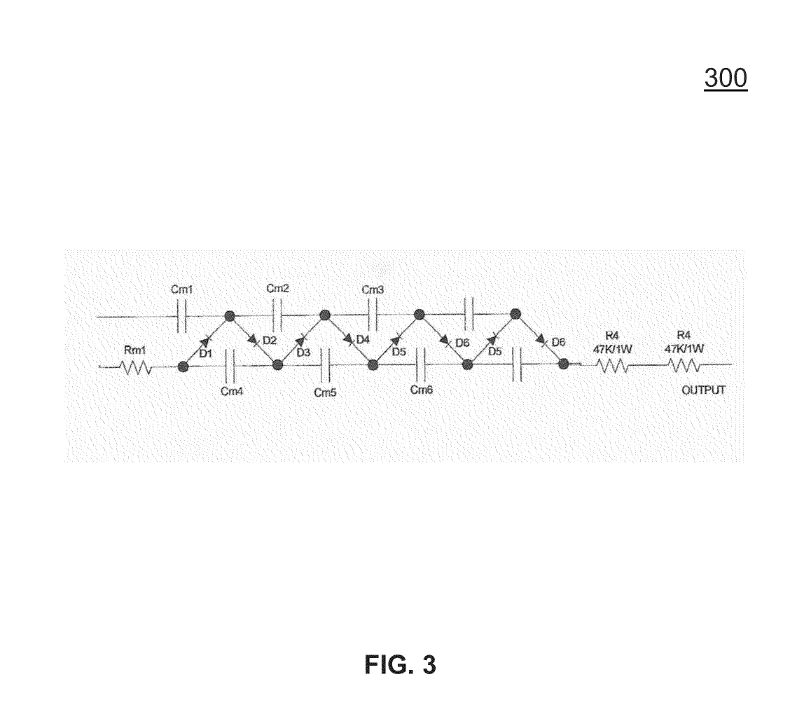

An alternative to relying on a transformer to provide the entire increase in output voltage is to use an add-on configurable circuit that is coupled to the output of the transformer-based circuit. FIG. 3 illustrates a schematic drawing of such an add-on configurable electronic circuit for a power circuit for a cold plasma DBD deodorant device, in accordance with an embodiment of the present disclosure. A diode-capacitor ladder network is illustrated that permits the setting of an appropriate voltage level of the cold plasma bromhidrosis treatment device. The output resistors provide an output impedance that can provide load regulation when connected to the cold plasma device. In an exemplary embodiment of the configurable circuit, such a diode-capacitor ladder network can raise the voltage from an input 7.5 kV to 47 kV. In a further exemplary embodiment, the polarity of the output pulsed waveform can be reversed by a reversal of the polarity of the diodes in the ladder network. It is notable that the diode-capacitor ladder network can raise the voltage using a relatively compact and light weight circuit.

FIGS. 4A and 4B illustrate two schematic views of a cold plasma deodorant (bromhidrosis treatment) device, in accordance with an embodiment of the present disclosure. The combination of the high voltage electrode together with the adjacent dielectric (e.g., smooth coated surface) constitutes a DBD device. FIG. 4A illustrates a cold plasma DBD deodorant device having an application surface 430, in accordance with an embodiment of the present disclosure. Application surface 430 is applied to treatment area (represented by capacitance 440), which is coupled to ground 450. In this implementation, a DC voltage source 410 provides input energy to the circuit. The DC power supply can be supplied by any means, including battery, AC/DC adapter and the like. The DC voltage (and the illustration of the batteries) is merely exemplary and not limiting in the choice of DC input voltage, or its particular implementation. An oscillator circuit is coupled to the DC power supply and the primary winding of a resonance transformer 420. The plan view in FIG. 4B illustrates that the smooth, coated surface 460 contacts the user. The surface 460 can be a smooth convex surface. Thus, the smooth coated surface acts like an insulator cladding that surrounds the high voltage electrode, such that application of the cold plasma can proceed safely. Hence, the smooth, coated surface is a dielectric surface that can be constructed using any of a number of suitable materials, such as crystalline quartz, PTFE, polyoxymethylene, and the like. These materials are merely exemplary, and not limiting as to the scope of materials that can be used for the coated surface in embodiments of the present disclosure. Beneath the dielectric surface is an underlying conductive electrode having sufficient capacitance to support the dielectric discharge of the DBD device. The gas can be a halogen gas, a noble gas or any other suitable gas capable of generating a cold plasma.

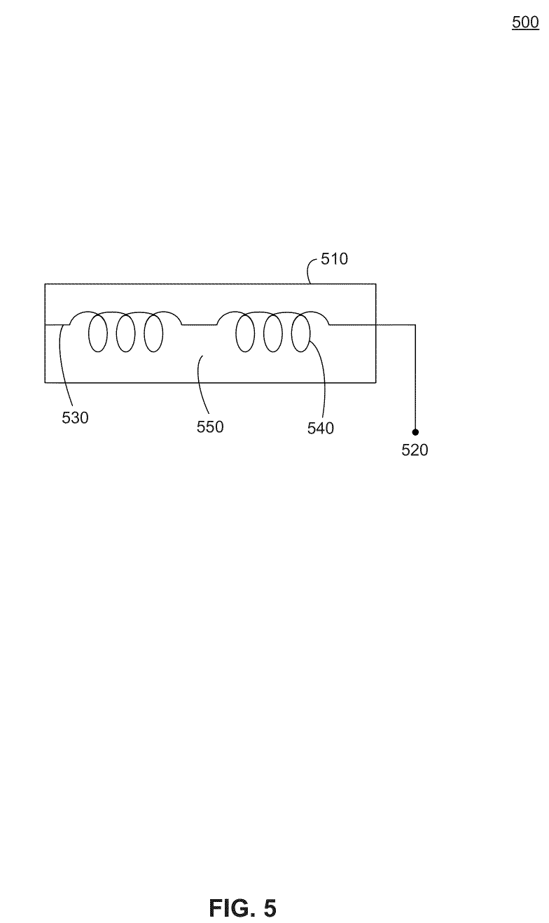

FIG. 5 illustrates a schematic of an electrode assembly of a cold plasma DBD deodorant device, in accordance with an embodiment of the present disclosure. FIG. 5 illustrates a cold plasma deodorant device embodiment having a quartz (or glass) containment tube 510 filled with a fluid 550 and a conductor 530, such as a tungsten filament wire. Fluid 550 may be any one of a noble gas, a halogen gas or a saline solution. Other gases and conductive solutions may also be used for fluid 550. Conductor 530 is coupled via port 520 to the high voltage output of the power circuit illustrated in FIG. 2. Conductor 530 optionally may contain coils 540 that provide the opportunity for more conductive material in the same volumetric space. As noted above, conductor 530 may be made of any suitable material such as tungsten wire or any other suitable conductor. In an exemplary embodiment, the tungsten wire can be 9 mil in diameter. Such a diameter is merely exemplary and not limiting in terms of the scope of the present disclosure.

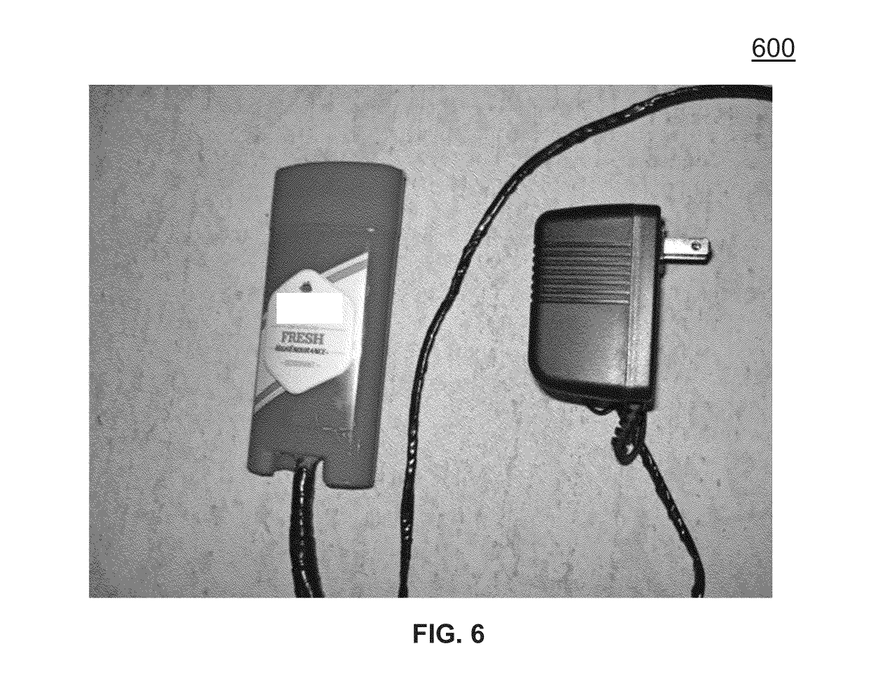

FIG. 6 is a photographic illustration of a cold plasma DBD deodorant device, in accordance with an embodiment of the present disclosure. This embodiment receives its power from being plugged into a wall outlet as opposed to other embodiments which can be battery powered, or powered by other means. In an additional embodiment, the cold plasma DBD deodorant device is rechargeable, and may be plugged into a wall outlet for that purpose. In a still further embodiment, the pulsed high voltage electrical signal may be generated at the wall outlet adaptor, and the pulsed high voltage electrical signal supplied via high voltage cable to the cold plasma DBD deodorant device.

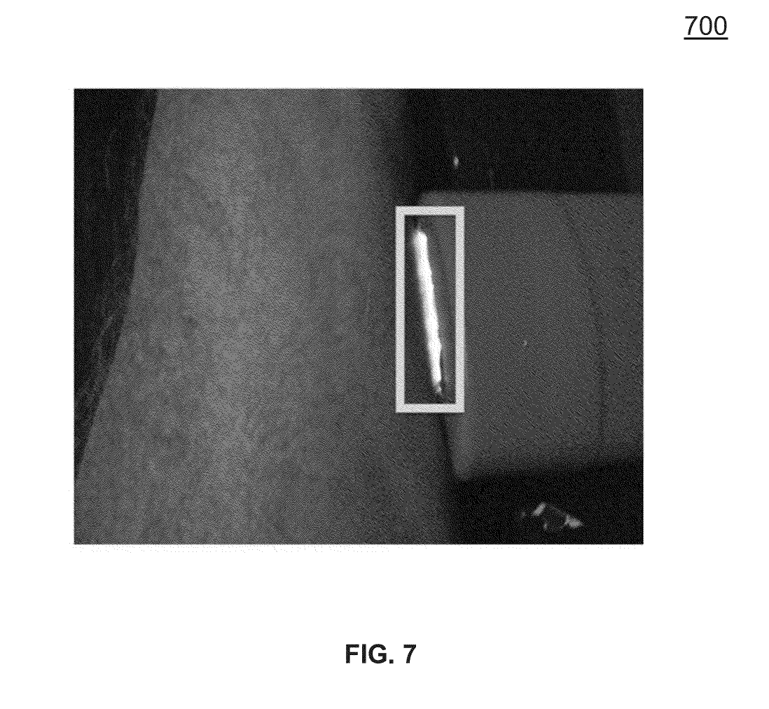

FIG. 7 illustrates a cold plasma DBD deodorant device in use, in accordance with an embodiment of the present disclosure. The box highlights the non-thermal plasma as it is generated from the DBD electrode of the device and conducted to the patient's skin.

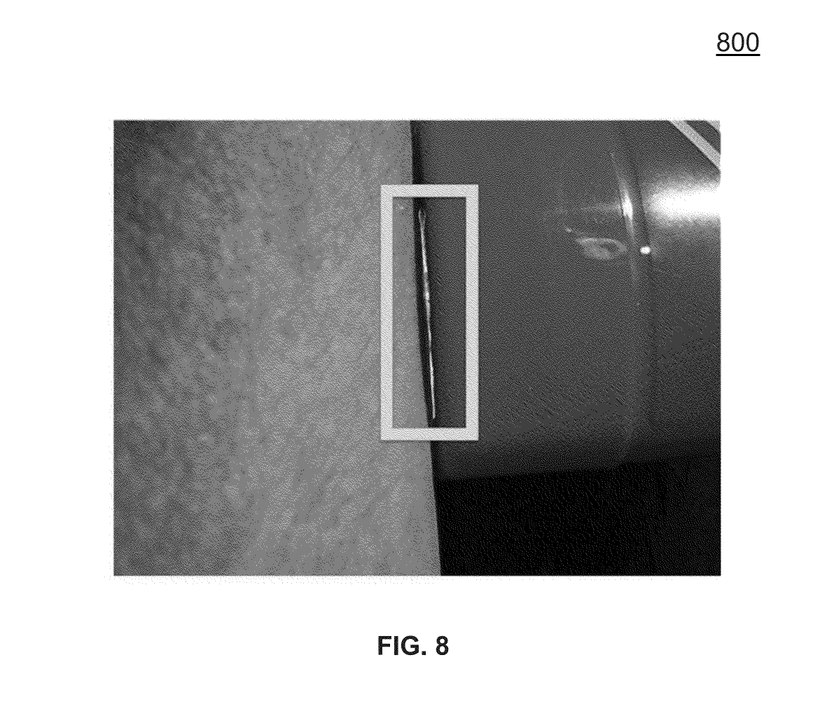

FIG. 8 provides a further illustration of a cold plasma DBD deodorant device in use, in accordance with an embodiment of the present disclosure. The box highlights the non-thermal plasma as it is generated from the DBD electrode of the device and conducted to the patient's skin.

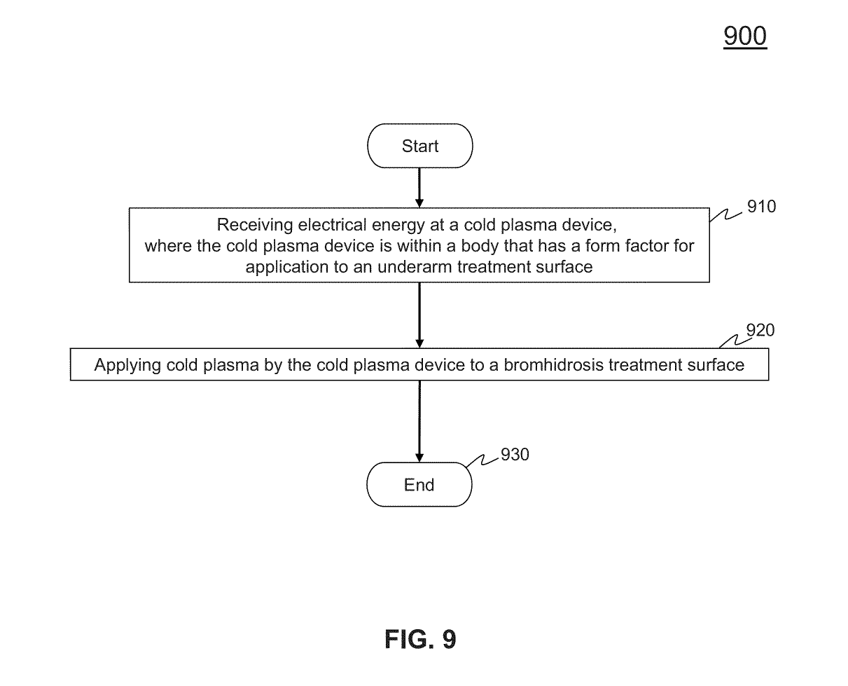

FIG. 9 provides a flowchart of a method for providing bromhidrosis treatment using a cold plasma device, according to an embodiment of the current invention.

The process begins at step 910. In step 910, electrical energy is received at a cold plasma device, where the cold plasma device is located within a body, the body having a form factor to facilitate application to an underarm treatment surface.

In step 920, cold plasma is applied by the cold plasma device to a bromhidrosis treatment surface.

At step 930, method 900 ends.

In summary, various embodiments of this disclosure refer to the approach of applying a cold plasma to the underarm area for deodorant purposes. The cold plasma may be generated by many means, such as a DBD device having a multitude of different possible electrode designs. Certain embodiments have a deodorant-like form factor for ready application to the underarm area. In addition, cold plasma may be generated by a gas jet plasma approach, such as that described in U.S. Provisional Application No. 60/913,369, filed Apr. 23, 2007; U.S. patent application Ser. No. 12/038,159, filed Feb. 27, 2008 (which issued as U.S. Pat. No. 7,633,231); and U.S. patent application Ser. No. 13/620,118, filed Sep. 14, 2012, all of which are incorporated by reference in their entireties. However, as noted earlier, other gas jet approaches may be used here (other than those described in the above cited applications) since the benefit of a multi-frequency harmonic-rich power supply (namely to power large cold plasma DBD device electrodes is not required for the cold plasma deodorant device.

As can be understood by one of ordinary skill in the art, the scope of the disclosure includes all methods of producing cold plasma. Included within the scope of this disclosure are the various cylindrical DBD electrodes and wand-like DBD electrodes that are discussed in Attorney Docket No. 3022.0070001, entitled “Method and Apparatus for Dielectric Barrier Discharge Wand Cold Plasma Device,” to be filed on Dec. 31, 2013, the disclosure of which is included by reference herein in its entirety.

The above embodiments describe materials that are exemplary and not limiting to the scope of various embodiments of the present disclosure. Thus, for examples, conductors other than tungsten may also be used for the electrode in various embodiments of the present disclosure. In addition, the above description refers to the use of a cold plasma, but also includes the use of a multi-frequency harmonic-rich cold plasma. Finally, the shape of the electrode can be configured to be compatible with the particular surface for which bromhidrosis treatment is desired to thereby ease the application of the cold plasma.

It is to be appreciated that the Detailed Description section, and not the Summary and Abstract sections, is intended to be used to interpret the claims. The Summary and Abstract sections may set forth one or more but not all exemplary embodiments of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the following claims and their equivalents.

What is claimed is:

1. A cold plasma bromhidrosis treatment device comprising:
a dielectric barrier discharge (DBD) device formed by an electrode disposed adjacent to a dielectric barrier, the dielectric barrier configured to apply a cold plasma to a bromhidrosis treatment surface; and
a body configured to hold the DBD device, the body having a form factor to facilitate application to an underarm treatment surface.

2. The cold plasma bromhidrosis treatment device of claim 1, wherein the body is further configured to receive a pulsed high voltage signal from an external cold plasma power supply, and configured to couple the received pulsed high voltage signal to the electrode.

3. The cold plasma bromhidrosis treatment device of claim 1, further comprising:
a housing formed within the body, the housing configured to accommodate a pulsed high voltage cold plasma power supply, and one or more batteries for connection to the pulsed high voltage cold plasma power supply.

4. The cold plasma bromhidrosis treatment device of claim 3, further comprising:
an AC/DC adaptor configured to couple the pulsed high voltage cold plasma power supply to an AC outlet.

5. The cold plasma bromhidrosis treatment device of claim 1, wherein the dielectric barrier comprises at least one of PTFE and polyoxymethylene.

6. The cold plasma bromhidrosis treatment device of claim 1, wherein a shape of the dielectric barrier is a smooth convex surface.

7. The cold plasma bromhidrosis treatment device of claim 1, wherein the pulsed high voltage cold plasma power supply comprises a transformer.

8. The cold plasma bromhidrosis treatment device of claim 1, wherein the pulsed high voltage cold plasma power supply comprises a diode-capacitance ladder network.

9. The cold plasma bromhidrosis treatment device of claim 1, wherein the dielectric barrier comprises a quartz containment tube including a fluid, the fluid being at least one of a noble gas, a halogen gas, or a saline solution.

10. The cold plasma bromhidrosis treatment device of claim 1, wherein the electrode comprises a substantially flat or gently curved shape, the electrode being covered with polyoxymethylene.

11. A method of bromhidrosis treatment comprising:
receiving, by a dielectric barrier discharge (DBD) device, electrical energy to generate a cold plasma, the dielectric barrier discharge (DBD) device formed by an electrode disposed adjacent to a dielectric barrier, and a body configured to hold the DBD device, the body having a form factor to facilitate application to an underarm treatment surface; and
applying the cold plasma by the dielectric barrier to a bromhidrosis treatment surface.

12. The method of claim 11, wherein the receiving electrical energy further includes:
receiving a pulsed high voltage signal from an external cold plasma power supply, and coupling the received pulsed high voltage signal to the electrode.

13. The method of claim 11, wherein the receiving electrical energy further includes:
receiving electrical energy from one or more batteries included in a housing formed within the body, for coupling to a pulsed high voltage cold plasma power supply within the housing.

14. The method of claim 11, wherein the receiving electrical energy further includes:
receiving electrical energy from an AC/DC adaptor configured to be coupled between the pulsed high voltage cold plasma power supply and an AC outlet.

15. The method of claim 11, wherein the dielectric barrier comprises at least one of PTFE and polyoxymethylene.

16. The method of claim 11, wherein a shape of the dielectric barrier is a smooth convex surface.

17. The method of claim 11, wherein the pulsed high voltage cold plasma power supply comprises a transformer.

18. The method of claim 11, wherein the pulsed high voltage cold plasma power supply comprises a diode-capacitance ladder network.

19. The method of claim 11, wherein the dielectric barrier comprises a quartz containment tube including a fluid, the fluid being at least one of a noble gas, a halogen gas, or a saline solution.

20. The method of claim 11, wherein the electrode comprises a substantially flat or gently curved shape, the electrode being covered with polyoxymethylene.

21. A method of bromhidrosis treatment comprising:
receiving, by a cold plasma device, electrical energy to generate a cold plasma, the cold plasma device being located with a body, the body having a form factor to facilitate application to an underarm treatment surface; and
applying the cold plasma by the cold plasma device to a bromhidrosis treatment surface.
